(19) United States
(12) Patent Application Publication
(10) Pub. No.: US 2003/0224344 A1
(43) Pub. Date: Dec. 4, 2003

(54) Method and system for clustering data

(76) Inventors: Ron Shamir, Rehovot (IL); Roded Sharan, Reut (IL)

Correspondence Address: Mark M Friedman, Bill Polkinghorn, Discovery Dispatch, 9003 Florin Way, Upper Marlboro, MD, 20772, US

(21) Appl. No.: 10/221,476

(22) PCT Filed: Mar. 13, 2001
(86) PCT No.: PCT/US01/07824

(30) Foreign Application Priority Data
Mar. 27, 2000 (US) 60192391

(51) Int. Cl. C12Q 1/00; C12Q 1/68; G06F 19/00; G01N 33/48; G01N 33/50; G06F 7/00; G06F 17/00

(52) U.S. Cl. 435/4; 702/19; 707/101

(57) ABSTRACT

A method of classifying a plurality of elements, such as genes, an associated system and an associated computer-read-able storage medium. Similarity values for pairs of elements are measured. For example, in the case of genes, gene expression fingerprints are measured, and the similarity values are computed from the fingerprints. A graph is constructed such that each vertex of the graph corresponds to a respective element. Each edge of the graph is assigned a superbinary weight that is based on the corresponding similarity value. The graph is partitioned into kernels, and the kernels are merged into clusters. Preferably, the superbinary weights are based on the similarity values according to a probabilistic model. The system of the present invention includes an apparatus for measuring the similarity values, a memory for storing the similarity values, and a proccesor for implementing the method of the present invention. The storage medium of the present invention includes computer readable code in which the method of the present invention is encoded.

### FIELD AND BACKGROUND OF THE INVENTION

The present invention relates to a method and system for determining or gathering information in the form of a data set, in which the distribution of the data set is not known or available, and for processing the data set to find a partition of the data set into several groups, or clusters, each group indicating the presence of a distinct category of the determined or gathered information.

Clustering is an important known technique in exploratory data analysis, where a priori knowledge of the distribution of the observed data is not available. Known prior art partitional clustering methods, that divide the data according to natural classes present therein, have been used in a large variety of scientific disciplines and engineering applications, including, among others, pattern recognition, learning theory, astrophysics, medical image and data processing, image compression, satellite data analysis, automatic target recognition, and speech recognition. See, for example, U.S. Pat. No. 5,706,503, to Poppen et al., and U.S. Pat. No. 6,021,383, to Domany et al., both of which patents are incorporated by reference for all purposes as if fully set forth herein.

Applications of clustering in molecular biology include the analysis of gene expression data, the analysis of protein similarity data, supervised and unsupervised gene and tissue classification, protein expression class discovery and identification of regulatory sequence elements.

Recently developed DNA microarray technologies enable the monitoring of expression levels of thousands of genes simultaneously. This allows for the first time a global view on the transcription levels of many (or all) genes when the cell undergoes specific conditions or processes. The potential of such technologies for functional genomics is tremendous: measuring gene expression levels in different developmental stages, different body tissues, different clinical conditions and different organisms is instrumental in understanding genes function, gene networks, biological processes and effects of medical treatments.

A key step in the analysis of gene expression data is the identification of groups of genes that manifest similar expression patterns over several conditions. The corresponding algorithmic problem is to cluster multi-condition gene expression patterns. The grouping of genes with similar expression patterns into clusters helps in unraveling relations between genes, deducing the function of genes and revealing the underlying gene regulatory network. Grouping of conditions with similar expression profiles can indicate disease types and treatment responses.

A clustering problem consists of n elements and a characteristic vector for each element. In gene expression data, elements are genes, and the vector of each gene contains its expression levels under some conditions. These levels are obtained by measuring the intensity of hybridization of gene-specific oligonucleotides (or cDNA molecules), which are immobilized to a surface, to a labeled target RNA mixture. A measure of pairwise similarity is then defined between such vectors. For example, similarity can be measured by the correlation coefficient between vectors. The goal is to partition the elements into subsets, which are called clusters, so that two criteria are satisfied: homogeneity—elements in the same cluster are highly similar to each other; and separation—elements from different clusters have low similarity to each other. As noted above, this problem has numerous applications in biology as well as in other disciplines.

There is a very rich literature on cluster analysis going back over three decades. Several algorithmic techniques were previously used in clustering gene expression data, including hierarchical clustering (M. B. Eisen et al., “Cluster analysis and display of genome-wide expression patterns”, PNAS vol. 95 pp. 14863-14868 (1998)), self organizing maps (P. Tamayo et al., “Interpreting patterns of gene expression with self-organizing maps: methods and application to hematopoietic differentiation”, PNAS vol. 96 pp. 2907:2912 (1999)), simulated annealing (U. Alon et al., “Broad patterns of gene expression revealed by clustering analysis of tumor and normal colon tissues probed by oligonucleotide arrays”, PNAS vol. 96 pp. 6745-6750 (1999)), and graph theoretic approaches (E. Hartuv et al., “An algorithm for clustering cDNAs for gene expression analysis using short oligonucleotide fingerprints”, Geonomics vol. 66 pp. 249-256 (2000); A. Ben-Dor et al., “Clustering gene expression patterns”, Journal of Computational Biology vol. 6 no. 3/4 pp. 281-297 (1999)).

Let N={e₁, . . . , eₙ} be a set of n elements and let C=(C₁, . . . , C₁) be a partition of N into l subsets. That is, the subsets are disjoint and their union is N. Each subset is called a cluster, and C is called a clustering solution, or simply a clustering. Two elements eᵢ and eⱼ are called mates with respect to C if they are members of the same cluster in C. In the gene expression context, the elements usually are the genes, and it often is assumed that there exists some correct partition of the genes into “true” clusters. When C is the true clustering of N, elements that belong to the same true cluster are simply called mates.

The input data for a clustering problem is typically given in one of two forms: (1) Fingerprint data—each element is associated with a real-valued vector, called the fingerprint or pattern of the element, which contains p measurements on the element, e.g., expression levels of an mRNA at different conditions, or hybridization intensities of a cDNA with different oligonucleotides. (2) Similarity data—pairwise similarity values between elements. Similarity values can be computed from fingerprint data, e.g. by correlation between vectors. Alternatively, the data can represent pairwise dissimilarity, e.g. by computing distances. Fingerprints contain more information than similarity. Given the fingerprints, it is possible to compute any chosen pairwise similarity function between elements. Moreover, many other computations are possible, e.g., computing the mean vector for a group of elements. Nevertheless, similarity is completely generic and can be used to represent the input to clustering in any application. There also is a practical consideration regarding the representation: the fingerprint matrix is of order n×p while the similarity matrix is of order n×n, and in gene expression applications, often n>>p.

The goal in a clustering problem is to partition the set of elements N into homogeneous and well-separated clusters. That is, elements from the same cluster should be highly similar to each other, while elements from different clusters should have low similarity to each other. Note that this formulation does not define a single optimization problem. Homogeneity and separation can be defined in many ways, leading to a variety of optimization problems. Even when homogeneity and separation are precisely defined, they define two conflicting objectives: the higher the homogeneity, the lower the separation, and vice versa.

Clustering problems and algorithms often are represented in graph-theoretic terms. Therefore, some basic graph-theoretic definitions now will be presented.

Let G=(V,E) be a graph. V represents the vertex set of G. E represents the edge set of G. The vertex set V of G also is denoted V(G). If a weight is assigned to each of the edges of G, then G is a weighted graph. For a subset R⊂V, the subgraph induced by R, denoted G_{R}, is obtained from G by deleting all vertices not in R and the edges incident on them. That is, G_{R}=(R,E_{R}) where E_{R}={(i,j)εE|i,jεR}. For a vertex νεV, the degree of ν is defined as the number of edges incident on ν, and the weight of ν is defined as the sum of the weights of the edges incident on ν. A cut Γ in G is a subset of the edges of G whose removal disconnects G. The weight of Γ is the sum of the weights of the edges of Γ. A minimum cut is a cut in G with a minimum number of edges. A minimum weight cut is a cut in G with minimum weight. If the edge weights all are non-negative, then a minimum weight cut Γ partitions the vertices of G into two disjoint non-empty subsets A,B⊂V, A∪B=V, such that E∩{(u,ν):uεA,νεB}=Γ. For a pair of vertices u,νεV, the distance between u and ν is the length of the shortest path which connects u and ν. Path length is measured by counting edges. The diameter of G is the maximum distance between a pair of vertices in G.

For a set of elements K⊂N, the fingerprint or centroid of N is defined as the mean vector of the fingerprints of the members of K. For two fingerprints x and y of two different elements, or of two different sets of elements, the similarity of the fingerprints is denoted by S(x,y). A similarity graph is a weighted graph in which vertices correspond to elements and edge weights are derived from the similarity values between the corresponding elements. Hence, the similarity graph is a representation of the similarity matrix.

Several methods of solving the clustering problem, for example, hierarchical algorithms and K-means algorithms, are known in the art. See, for example, R. Shamir and R. Sharan, “Algorithmic approaches to clustering gene expression data”, to appear in Current Topics in Computational Biology, T. Jiang et al. eds., MIT Press.

The algorithm of E. Hartuv et al., “An algorithm for clustering cDNA fingerprints”, Geonomics vol. 66 no. 3 pp. 249-256 (2000)) uses a graph theoretic approach to clustering. The input data is represented as an unweighted similarity graph in which there is an edge between two vertices if and only if the similarity between their corresponding elements exceeds a predefined threshold. The algorithm recursively partitions the current set of elements into two subsets. Before a partition, the algorithm considers the subgraph induced by the current subset of elements. If the subgraph satisfies a stopping criterion then the subgraph is designated as a cluster. Otherwise, a minimum cut is computed in that subgraph, and the vertex set is split into the two subsets separated by that cut. The output is a list of clusters. This scheme is defined recursively in FIG. 1 as a procedure called “FormClusters”. Procedure MinCut(G) computes a minimum cut of G and returns a partition of G into two subgraphs H and H′ according to this cut.

The following notion is key to the algorithm: A highly connected subgraph (HCS) is an induced subgraph H of G whose minimum cut value exceeds |V(H)|/2, that is, H remains connected if any └|V(H)|/2540 of its edges are removed. The algorithm identifies highly connected subgraphs as clusters. FIG. 2 demonstrates an application of this algorithm.

To improve separation in practice, several heuristics are used to expand the clusters and speed up the algorithm, as follows:

First, several (1 to 5) HCS iterations are carried out until no new cluster is found. Singletons can be “adopted” by clusters. For each singleton element x, the number of neighbors it has in each cluster and in the singleton set S is computed. If the maximum number of neighbors is sufficiently large, and is obtained by one of the clusters (rather than by S), then x is added to that cluster. The process is repeated several times.

When the similarity graph includes vertices of low degree, one iteration of the minimum cut algorithm may simply separate a low degree vertex from the rest of the graph. This is computationally very expensive, not informative in terms of the clustering, and may happen many times if the graph is large. Removing low degree vertices from G eliminates such iterations, and significantly reduces the running time. The process is repeated with several thresholds on the degree.

To overcome the possibility of cluster splitting, a final cluster-merging step is applied. This step uses the raw fingerprints. An average fingerprint is computed for each cluster, and clusters that have highly similar fingerprints are merged.

The algorithm of Hartuv et al. is based on a very simple unweighted graph, or, equivalently, on a graph in which the weights of present edges all are 1 and the weights of missing edges all are 0. Intuition suggests that better results could be obtained using real weights, or, at the very least, weights that can take on more than two possible values. There is thus an obvious need for, and it would be highly advantageous to have, a graph-theoretic clustering algorithm that uses weights that can take on more than two possible values.

### SUMMARY OF THE INVENTION

According to the present invention there is provided a method of classifying a plurality of elements, including the steps of: (a) for each pair of elements, measuring a respective similarity value; (b) partitioning a graph, each vertex whereof corresponds to a respective element, and each edge whereof is assigned a superbinary weight that is based on the similarity value of the pair of elements corresponding to the vertices that are connected by that edge, into a plurality of kernels, according to the weights; and (c) merging the kernels into clusters.

According to the present invention there is provided a system for classifying a plurality of elements, including: (a) an apparatus for measuring, for each pair of elements, a corresponding similarity value; (b) a memory for storing the similarity values; and (c) a processor for: (i) partitioning a graph, each vertex whereof corresponds to a respective element, and each edge whereof is assigned a superbinary weight that is based on the similarity value of the pair of elements corresponding to the vertices that are connected by that edge, into a plurality of kernels, according to the weights, and (ii) merging the kernels into clusters.

According to the present invention there is provided a system for classifying a plurality of elements, including: (a) an apparatus for measuring, for each element, a respective fingerprint; (b) a memory for storing the fingerprints; and (c) a processor for: (i) computing, for each pair of elements, from the fingerprints thereof, a corresponding similarity value, (ii) partitioning a graph, each vertex whereof corresponds to a respective element, and each edge whereof is assigned a superbinary weight that is based on the similarity value of the pair of elements corresponding to the vertices that are connected by that edge, into a plurality of kernels, according to the weights, and (iii) merging the kernels into clusters.

According to the present invention there is provided a method for analyzing signals containing a data set which is representative of a plurality of physical phenomena, to identify and distinguish among the physical phenomena by determining clusters of data points within the data set, the method including the steps of: (a) associating a similarity value with each pair of data points; (b) partitioning a graph, each vertex whereof corresponds to a respective data point, and each edge whereof is assigned a superbinary weight that is based on the similarity value of the pair of data points corresponding to the vertices that are connected by that edge, into a plurality of kernels, according to the weights; (c) merging the kernels to form the clusters; and (d) identifying the physical phenomena based on the data clusters.

According to the present invention there is provided an apparatus for analyzing signals containing a data set which is representative of a plurality of physical phenomena, to identify and distinguish among the physical phenomena by determining clusters of data points within the data set, including: (a) a mechanism for associating, with each pair of data points, a corresponding similarity value; (b) a mechanism for partitioning a graph, each vertex whereof corresponds to a respective data point, and each edge whereof is assigned a superbinary weight that is based on the similarity value of the pair of data points corresponding to the vertices that are connected by that edge, into a plurality of kernels, according to the weights; and (c) a mechanism for merging the kernels to form the clusters.

According to the present invention there is provided a computer readable storage medium having computer readable code embodied on the computer readable storage medium, the computer readable code for clustering multi-dimensional related data in a computer database, the computer database including a set of data records, each data record storing information about a respective object of interest, the computer readable code including: (a) program code for computing a similarity value for each pair of data records; (b) program code for constructing a graph, each vertex whereof corresponds to a respective data record, and each edge whereof is assigned a superbinary weight that is based on the similarity value of the pair of data records corresponding to the vertices that are connected by that edge; (c) program code for partitioning the graph into a plurality of kernels according to the weights; and (d) program code for merging the kernels to form the clusters.

According to the present invention there is provided a computer readable storage medium having computer readable code embodied on the computer readable storage medium, the computer readable code for clustering multi-dimensional related data in a computer database, the computer database including a set of data records, each data record storing information about a respective object of interest, the computer database also including, for at least one pair of data records, a corresponding similarity value, the computer readable code including: (a) program code for constructing a graph, each vertex whereof corresponds to a respective data record, and each edge whereof is assigned a superbinary weight that is based on the similarity value of the pair of data records corresponding to the vertices that are connected by that edge; (b) program code for partitioning the graph into a plurality of kernels according to the weights; and (c) program code for merging the kernels to form the clusters.

According to the present invention there is provided a method of classifying a plurality of elements, including the steps of: (a) for each pair of elements, measuring a respective similarity value; (b) partitioning the elements into clusters according to the similarity values; (c) computing at least one figure of merit, for the partitioning, selected from the group consisting of: (i) at least one measure of a homogeneity of the clusters; and (ii) at least one measure of a separation of the clusters.

According to the present invention there is provided a method for analyzing signals containing a data set which is representative of a plurality of physical phenomena, to identify and distinguish among the physical phenomena by determining clusters of data points within the data set, the method including the steps of: (a) for each pair of elements, measuring a respective similarity value; (b) partitioning the elements into clusters according to the similarity values; (c) computing at least one figure of merit, for the partitioning, selected from the group consisting of: (i) at least one measure of a homogeneity of the clusters, and (ii) at least one measure of a separation of the clusters; and (d) identifying the physical phenomena based on the data clusters.

Although the present invention is described herein primarily in terms of its application to the clustering of gene expression data, it is to be understood that the scope of the present invention extends to all practical applications of clustering. As discussed above, these applications include, but are not limited to, pattern recognition, time series prediction, learning theory, astrophysics, medical applications including imaging and data processing, network partitioning, image compression, satellite data gathering, data base management, data base mining, data base analysis, automatic target recognition and speech and text recognition. To this end, the real world physical entities that are classified by the method of the present invention are called herein either “elements” or “physical phenomena” or “objects of interest”. The first step of the method of the present invention consists of measuring the properties of the elements that are to form the basis of the classification. For example, in the application of the present invention to the clustering of gene expression data, the first step is the measurement of gene fingerprints. Alternatively, and in particular in data base applications of the present invention, the data base is stored in an appropriate memory and includes a data set that is representative of related physical phenomena. Each data point of the data set is a description of one of the phenomena. A processor, that is programmed to effect the method of the present invention, receives, from the memory, signals representative of the data points. The processor then classifies the data points in accordance with the method of the present invention.

The present invention builds on the algorithm of Hartuv et al. by using a weighted similarity graph. The weights are based on measured or calculated similarity values for the physical phenomena that are to be classified, according to a probabilistic model of the physical phenomena being classified. Although the scope of the present invention includes the use of any suitable probabilistic distribution model, the preferred probabilistic model assumes that the similarity values are normally distributed. Preferably, the probabilistic model is parametrized according to one or more parameters, and one preliminary step of the classification is the estimation of these parameters. Most preferably, this estimation is based on a previously classified subset of the phenomena. Alternatively, the estimating is effected using an EM algorithm.

Like the algorithm of Hartuv et al., the method of the present invention recursively partitions connected components of the graph. The present invention adds to the algorithm of Hartuv et al. the concept of a kernel, as defined below. Specifically, and unlike the algorithm of Hartuv et al., the stopping criterion of the recursive partitioning is such that the subgraph is a kernel. Connected components that are not kernels are referred to herein as composite connected components, and each recursive partition divides a composite connected component into two subgraphs. Because this recursive partition produces two subgraphs, it is referred to herein as a “bipartition”. Thus, the output of the recursive partitioning is a list of kernels that serve as a basis for the clusters. The corresponding recursively-defined procedure, “FormKernels”, is illustrated in FIG. 3; the resemblance of FormKernels to the prior art procedure FormClusters of FIG. 1 is evident. The main difference between FormKernels and FormClusters is the substitution of the procedure MinWeightCut(G) for the procedure MinCut(G). MinWeightCut(G) computes a minimum weight cut of G and returns a partition of G into two subgraphs H and H′ according to this cut. Note that MinWeightCut(G) is a generalization of MinCut(G) to the case of “superbinary” weights. In the present context, a “superbinary” weight is a weight that can take on any value selected from a set of three or more members, for example, the set {0, 0.5, 1}. In this way, the present invention is distinguished from the algorithm of Hartuv et al., in which, in effect, the weights are allowed to assume only one of two possible values (zero or one). Preferably, the weights of the present invention can take on any value in any closed real interval, for example the interval [0,1].

Under some circumstances, as discussed below, it is preferable to substitute a minimum s-t cut for one or more of the minimum weight cuts.

After all the kernels have been constructed, the kernels are merged to form the clusters that constitute the output of the method of the present invention.

Preferably, after each round of bipartitions, each vertex that is a singleton (i.e., each vertex that is disconnected from the rest of the graph), and that is sufficiently similar to one of the kernels, is adopted into the kernel to which that vertex is most similar. Preferably a similar adoption step is effected subsequent to the merger of the kernels to form the clusters. For enhanced efficiency, it is preferable, prior to the bipartition of a large composite connected component, to screen the low weight vertices of the component.

The minimal input to the algorithm of the present invention is a set of similarity values (or, equivalently, dissimilarity or distance values) for all pairs of elements. Alternatively, and preferably, fingerprints of the elements are measured, and the similarity values are computed from the fingerprints. Preferably, the similarity values are inner products of the fingerprints.

The scope of the present invention also includes a computer readable storage medium in which is embodied computer readable code for implementing the algorithm of the present invention.

The scope of the present invention also includes innovative figures of merit for clustering solutions generally.

### BRIEF DESCRIPTION OF THE DRAWINGS

The invention is herein described, by way of example only, with reference to the accompanying drawings, wherein:

FIG. 1 (prior art) shows the FormClusters procedure of Hartuv et al.;

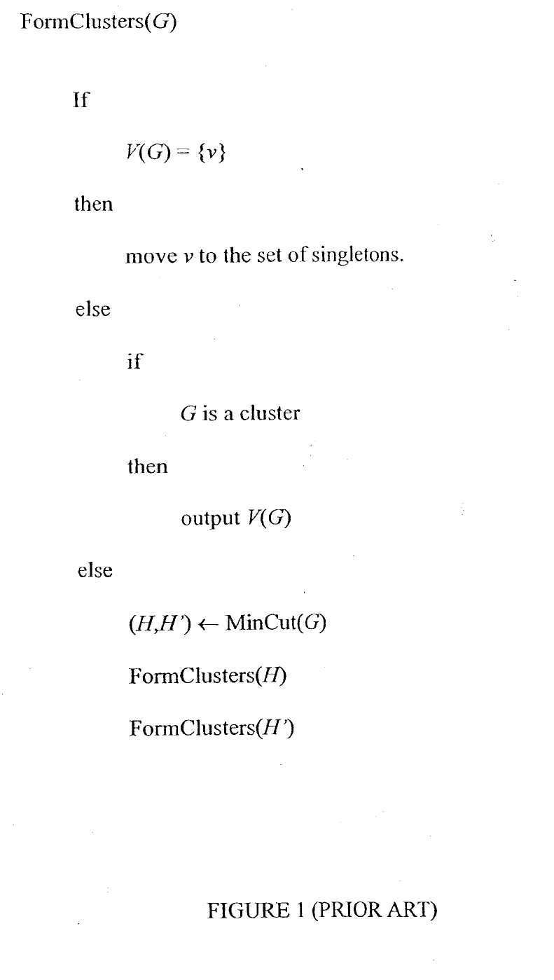

FIG. 2 (prior art) illustrates the clustering algorithm of Hartuv et al.;

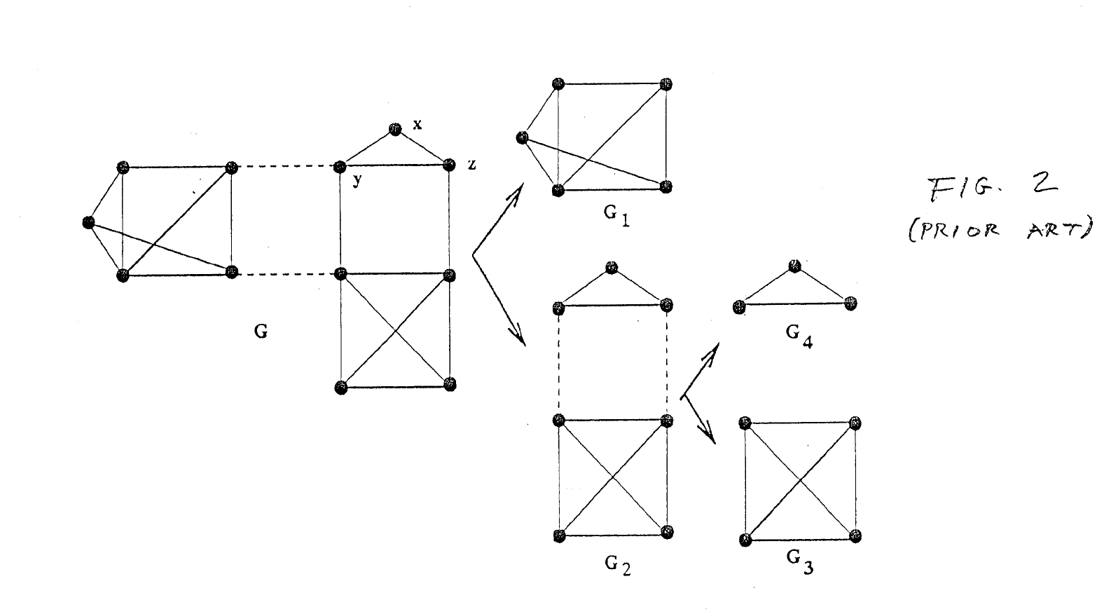

FIG. 3 shows the FormKernels procedure of the present invention;

FIG. 4 is a flowchart of the method of the present invention;

FIG. 5 shows how the method of the present invention clustered the gene expression data of Cho et al.;

FIG. 6 shows how the method of the present invention clustered the gene expression data of Iyer et al.;

FIG. 7 is a histogram of similarity values for cDNA oligonucleotide fingerprints;

FIG. 8 is a high level block diagram of a system of the present invention.

### DESCRIPTION OF THE PREFERRED EMBODIMENTS

The present invention is of a method and system of measuring characteristics of elements and classifying the elements according to the measured characteristics. Specifically, the present invention can be used to gather and cluster molecular biological data such as gene expression data.

The principles and operation of data clustering according to the present invention may be better understood with reference to the drawings and the accompanying description.

The analysis of the raw data involves three main steps: (1) Preprocessing—normalization of the data and calculation of pairwise similarity values between elements; (2) Clustering; and (3) Assessment of the results.

The goal of the preprocessing step is to normalize the data, define a similarity measure between elements and characterize mates and non-mates in terms of their pairwise similarity values.

Common procedures for normalizing fingerprint data include transforming each fingerprint to have mean zero and variance one, a fixed norm, a fixed maximum entry, etc. Choosing an appropriate procedure depends on the kind of data dealt with, and on the biological context of the study. Examples for different data normalization procedures are given below.

Often, each fingerprint in the normalized data has the same norm. If fixed-norm fingerprints are viewed as points in the Euclidean space, then these points lie on a p-dimensional sphere, and the inner product of two vectors is proportional to the cosine of the angle between them. Therefore, in that case, the vector inner product is the preferred similarity measure. In case all fingerprints have mean zero and variance one, the inner product of two vectors equals their correlation coefficient.

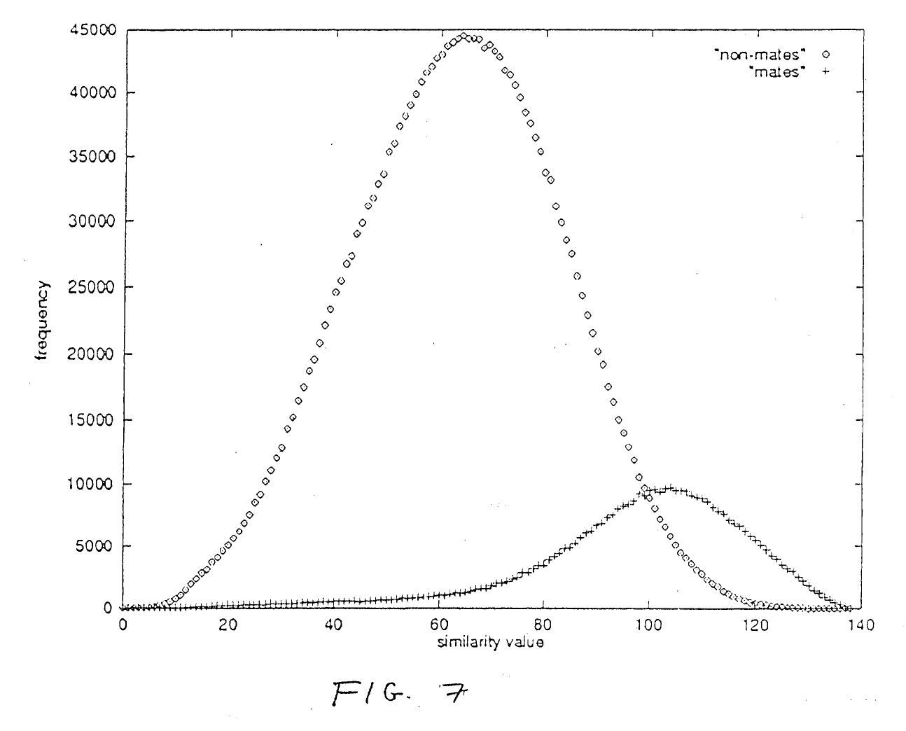

Preferably, according to the present invention, pairwise similarity values between elements are normally distributed: Similarity values between mates are normally distributed with mean μ_{T} and variance σ²_{T}, and similarity values between non-mates are normally distributed with mean μ_{F} and variance σ²_{F}, where μ_{T}>μ_{F}. This situation was observed on real data (for example, see FIG. 7 below), and can be theoretically justified by the Central Limit Theorem. ƒ(x|μ_{T},σ_{T}) denotes the mates probability density function. ƒ(x|μ_{F},σ_{F}) denotes the non-mates probability density function.

When similarity values are not normally distributed, then their distribution can be approximated, and the same ideas presented below can be applied. In practice, the normality assumption often holds, as demonstrated by the experimental results presented below.

An initial step of the algorithm is estimating the distribution parameters μ_{T}, μ_{F}, σ_{T} and σ_{F}, and the probability Pₘₐₜₑₛ that two randomly chosen elements are mates. There are two possible methods to compute these parameters: (1) In many cases the true partition for a subset of the elements is known. This is the case, for example, if the clustering of some of the genes in a cDNA chip experiment is found experimentally, or more generally, if a subset of the elements has been analyzed using prior biological knowledge. Based on this partition one can compute the sample mean and sample variance for similarity values between mates and between non-mates, and use these as maximum likelihood estimates for the distribution parameters. The proportion of mates among all pairs can serve as an estimate for Pₘₐₜₑₛ, if the subset was randomly chosen. (2) In case no additional information is given, these parameters can be estimated using the EM algorithm (see, e.g., B. Mirkin, Mathematical Classification and Clustering (Kluwer, 1996), pp. 154-155).

Let S be a pairwise similarity matrix for the fingerprint matrix M, where Sᵢⱼ is the inner product between the fingerprint vectors of the elements eᵢ and eⱼ, ie.,

$${S}_{i j} = {\sum }_{k = 1}^{p} {M}_{ik} {M}_{jk} .$$

The algorithm represents the input data as a weighted similarity graph G=(V,E). In this graph vertices correspond to elements and edge weights are derived from the similarity values. The weight wᵢⱼ of an edge (i,j) reflects the probability that i and j are mates, and is set to be

$${w}_{i j} = ln \frac{{p}_{mates} f ( {S}_{i j} \vert i , j are mates )}{( 1 - {p}_{mates} ) f ( {S}_{i j} \vert i , j are not mates )}$$

Here ƒSᵢⱼ|i,j are mates)=ƒSᵢⱼ|μ_{T},σ_{T}) is the value of the mates probability density function at Sᵢⱼ:

$$f ( {S}_{i j} \vert i , j are mates ) = \frac{1}{\sqrt{2 π} {σ}_{T}} exp ( - \frac{{( {S}_{i j} - {μ}_{T} )}^{2}}{2 {σ}_{T}^{2}} )$$

Similarly, ƒSᵢⱼ|i,j are non-mates) is the value of the non-mates probability density function at Sᵢⱼ:

$$\begin{matrix}f ( {S}_{i j} \vert i , j are non \text{-} mates ) = \frac{1}{\sqrt{2 π} {σ}_{F}} exp ( - \frac{{( {S}_{i j} - {μ}_{F} )}^{2}}{2 {σ}_{F}^{2}} ) \\ Hence , {w}_{i j} = ln \frac{{p}_{mates} {σ}_{F}}{( 1 - {p}_{mates} ) {σ}_{T}} + \frac{{( {S}_{i j} - {μ}_{F} )}^{2}}{2 {σ}_{F}^{2}} - \frac{{( {S}_{i j} - {μ}_{T} )}^{2}}{2 {σ}_{T}^{2}}\end{matrix}$$

For efficiency, low weight edges are omitted from the graph, so that there is an edge between two elements if and only if their pairwise similarity value is above some predefined non-negative threshold t_{S}.

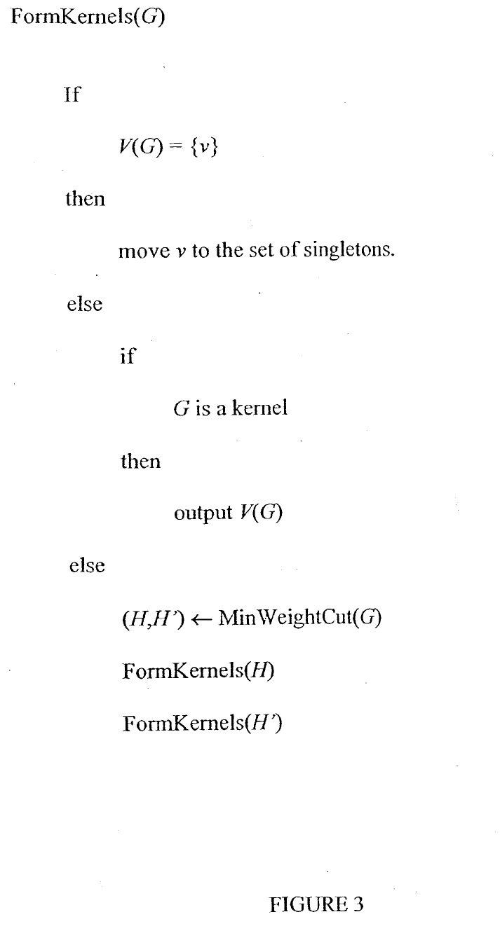

The basic procedure of the present invention, FormKernels, is illustrated in FIG. 3. FormKernels can be described recursively as follows: In each step the procedure handles some connected component of the subgraph induced by the yet-unclustered elements. If the component contains a single vertex, then this vertex is considered a singleton and is handled separately. Otherwise, a stopping criterion (which is described below) is checked. If the component satisfies the criterion, the component is declared a kernel. Otherwise, the component is split according to a minimum weight cut. The procedure outputs a list of kernels which serve as a basis for the eventual clusters. It is assumed that procedure MinWeightCut(G) computes a minimum weight cut of G and returns a partition of G into two subgraphs H and H′ according to this cut.

The idea behind FormKernels is the following. Given a connected graph G, the object is to decide whether V(G) is a subset of some true cluster, or V(G) contains elements from at least two true clusters. In the first case G is termed pure. In the second case, G is termed composite. In order to make this decision, the following two hypotheses are tested for each cut Γ in G:

H^{Γ}₀: Γ contains only edges between non-mates

H^{Γ}₁: Γ contains only edges between mates

If G is pure then H^{Γ}₁ is true for every cut Γ of G. If on the other hand G is composite, then there exists at least one cut Γ for which H^{Γ}₀ holds. Therefore, G is determined to be pure if and only if H^{Γ}₁ is accepted for each cut Γ in G. If G is found to be pure, G is declared to be a kernel. Otherwise, V(G) is partitioned into two disjoint subsets, according to a cut Γ in G for which the posterior probability ratio Pr(H^{Γ}₁|Γ)/Pr(H^{Γ}₀|Γ) is minimum. Here, Pr(H^{Γ}ᵢ|Γ) denotes the posterior probability for H^{Γ}ᵢ, i=0,1, given a cut Γ (along with its edge weights). Such a partition is called a weakest bipartition of G.

It first will be shown how to find a weakest bipartition of G. To this end, a simplifying probabilistic assumption is made: For a cut Γ in G the random variables {Sᵢⱼ}_{(i,j)εΓ} are mutually independent. The weight of a cut Γ is denoted by W(Γ). The likelihood that the edges of Γ connect only non-mates is denoted by ƒΓ|H^{Γ}₀). The likelihood that the edges of Γ connect only mates is denoted by ƒ(Γ|H^{Γ1}). Let Pr(H^{Γ}ᵢ) denote the prior probability for H^{Γ}ᵢ, i=0,1.

Lemma: Let G be a complete graph. Then for any cut Γ in G

$$W ( Γ ) = ln \frac{Pr ( {H}_{1}^{Γ} \vert Γ )}{Pr ( {H}_{0}^{Γ} \vert Γ )}$$

Proof: By Bayes Theorem,

$$\frac{Pr ( {H}_{1}^{Γ} \vert Γ )}{Pr ( {H}_{0}^{Γ} \vert Γ )} = \frac{Pr ( {H}_{1}^{Γ} ) f ( Γ \vert {H}_{1}^{Γ} )}{Pr ( {H}_{0}^{Γ} ) f ( Γ \vert {H}_{0}^{Γ} )}$$

The joint probability density function of the weights of the edges in Γ, given that these weights are normally distributed with mean μ_{T} and variance σ²_{T}, is

$$\begin{matrix}f ( Γ \vert {H}_{1}^{Γ} ) = {\prod }_{( i , j ) ∈ Γ}^{} \frac{1}{\sqrt{2 π} {σ}_{T}} exp ( - \frac{{( {S}_{i j} - {μ}_{T} )}^{2}}{2 {σ}_{T}^{2}} ) \\ Similarly , f ( Γ \vert {H}_{0}^{Γ} ) = {\prod }_{( i , j ) ∈ Γ}^{} \frac{1}{\sqrt{2 π} {σ}_{F}} exp ( - \frac{{( {S}_{i j} - {μ}_{F} )}^{2}}{2 {σ}_{F}^{2}} ) \end{matrix}$$

The prior probability for H^{Γ}₁ is (Pₘₐₜₑₛ)^{|Γ|}. The prior probability for H^{Γ}₀ is (1-Pₘₐₜₑₛ)^{|Γ|}.

Therefore

$$\begin{matrix}ln \frac{Pr ( {H}_{1}^{Γ} \vert Γ )}{Pr ( {H}_{0}^{Γ} \vert Γ )} = \frac{Pr ( {H}_{1}^{Γ} ) f ( Γ \vert {H}_{1}^{Γ} )}{Pr ( {H}_{0}^{Γ} ) f ( Γ \vert {H}_{0}^{Γ} )} \\ = \vert Γ \vert ln \frac{{p}_{mates} {σ}_{F}}{( 1 - {p}_{mates} ) {σ}_{F}} + {\sum }_{( i , j ) ∈ Γ}^{} \frac{{( {S}_{i j} - {μ}_{F} )}^{2}}{2 {σ}_{F {□}^{□}}^{2}} - \\ {\sum }_{( i , j ) ∈ Γ}^{} \frac{{( {S}_{i j} - {μ}_{T} )}^{2}}{2 {σ}_{T}^{2}} = W ( Γ )\end{matrix} \qquad QED$$

Suppose at first that G is a complete graph and no threshold was used to filter edges. From the Lemma it follows that a minimum weight cut of G induces a weakest bipartition of G.

It remains to show how to decide if G is pure, or equivalently, which stopping criterion to use. For a cut Γ, H^{Γ}₁ is accepted if and only if Pr(H^{Γ}₁|Γ)>Pr(H^{Γ}₀|Γ). That is, the hypothesis with higher posterior probability is accepted.

Let Γ be a minimum weight cut of G, which partitions G into two subgraphs H and H′. By the previous lemma, for every other cut Γ′ of G

$$ln \frac{Pr ( {H}_{1}^{Γ} \vert Γ )}{Pr ( {H}_{0}^{Γ} \vert Γ )} = W ( Γ ) \leq W ( {Γ}^{′} ) = ln \frac{Pr ( {H}_{1}^{{Γ}^{′}} \vert {Γ}^{′} )}{Pr ( {H}_{0}^{{Γ}^{′}} \vert {Γ}^{′} )}$$

Therefore, H^{Γ}₁ is accepted for Γ if and only if H^{Γ}₁ is accepted for every cut Γ′ in G. Thus, H_{Γ}₁ is accepted and G is declared a kernel if and only if W(Γ)>0.

These ideas now are extended to the case that G is incomplete. Consider first the decision whether G is pure or composite. It is now possible that H^{Γ}₁ will be accepted for Γ but rejected for some other cut of G. Nevertheless, a test based on W(Γ) approximates the desired test. In order to apply the test criterion, the contribution of the edges missing from Γ to the posterior probability ratio Pr(H^{Γ}₁|Γ)|Pr(H^{Γ}₀|Γ) must be estimated. This is done as follows: Let F=(H×H)\Γ and let r=|H∥H′|. Denote by Φ(•) the cumulative standard normal distribution function. Define

$$\begin{matrix}{W}^{*} ( Γ ) ≡ ln \frac{{\prod }_{( i , j ) ∈ F}^{} {p}_{mates} Pr ( {S}_{i j} \leq {t}_{s} \vert {H}_{1}^{Γ} )}{{\prod }_{( i , j ) ∈ F}^{} ( 1 - {p}_{mates} ) Pr ( {S}_{i j} \leq {t}_{s} \vert {H}_{0}^{Γ} )} \\ = ( r - \vert Γ \vert ) ln \frac{{p}_{mates} Φ ( ( {t}_{S} - {μ}_{T} ) / {σ}_{T} )}{( 1 - {p}_{mates} ) Φ ( ( {t}_{S} - {μ}_{T} ) / {σ}_{F} )}\end{matrix}$$

G is declared to be a kernel if and only if W(Γ)+W*(Γ)>0.

In case it is decided that G is composite, Γ is used in order to partition G into two components. This yields an approximation of a weakest bipartition of G.

Because we are interested in testing H^{Γ}₀ and H^{Γ}₁ on a specific minimum weight cut Γ, the contribution of the missing edges to the posterior probability ratio can be calculated accurately by computing the real weights of the missing edges from the raw data. This of course increases the running time of the procedure.

Optionally, to ensure the tightness of the kernels, it is required that the diameter of each kernel be at most 2. This constraint holds with high probability for true clusters that are sufficiently large.

FormKernels produces kernels of clusters, which should be expanded to yield the full clusters. The expansion is done by considering the singletons which were found during the iterative execution of FormKernels. We denote by L and R the current lists of kernels and singletons, respectively. An adoption step repeatedly searches for a singleton ν and a kernel K whose pairwise fingerprint similarity is maximum among all pairs of singletons and kernels (in practice we consider only kernels with at least five members). If the value of this similarity exceeds some predefined threshold, then ν is adopted to K, that is, ν is added to K and removed from R, and the fingerprint of K is updated. Otherwise, the iterative process ends.

The main advantage of this approach is that adoption is decided based on the full raw data M, i.e., on the fingerprints, in contrast to other approaches in which adoption is decided based on the similarity graph.

After the adoption step takes place, a recursive clustering process is started on the set R of remaining singletons. This is done by discarding all other vertices from the initial graph. This iteration continues until no change occurs.

The penultimate step of the method of the present invention is a merging step: clusters whose fingerprints are similar are merged. The merging is done iteratively, each time merging two kernels whose fingerprint similarity is the highest, provided that this similarity exceeds a predefined threshold. When two kernels are merged, these kernels are removed from L, the newly merged kernel is added to L, and the fingerprint of the newly merged kernel is calculated. Finally, a last singleton adoption step is performed.

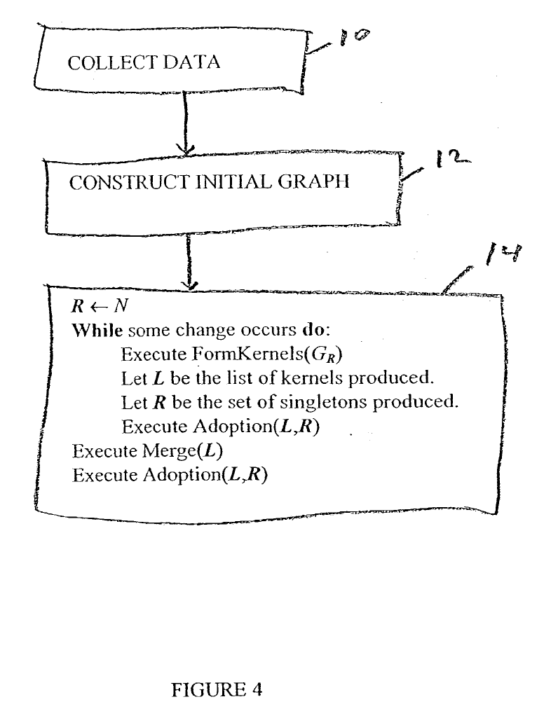

FIG. 4 is a flow chart of the overall method of the present invention. In box 10, the data, for example, gene expression fingerprints, are collected. In box 12, the initial graph G is constructed, for example by steps including computing the similarities of the fingerprints. In box 14, the algorithm of the present invention is executed. The first step of the algorithm is the initialization of the set R of unclustered elements to include all the elements of N. G_{R} is the subgraph of G induced by the vertex set R. Procedure Adoption(L,R) performs the singleton adoption step. Procedure Merge(L) performs the merging step.

If the input to the algorithm of the present invention is similarity data rather than fingerprint data, then the adoption and merging steps must be modified. For the adoption, each singleton s is tested against each kernel K. Let H be the subgraph of G induced by V(K)∪{s}. Let Γ be the cut in H which is induced by the partition (V(K),{s}). The value W(Γ)+W*(Γ) is computed and is used to score the correspondence between s and K. In each adoption iteration, the pair s,K with the highest correspondence score is chosen, and K adopts s if this score exceeds a predefined threshold. The merging step is modified similarly. For any two clusters K₁ and K₂, the relevant cut is the cut Γ=(K₁,K₂) in the subgraph induced by V(K₁)∪V(K₂).

Two ad-hoc refinements, screening and minimum s-t cuts, were developed in order to reduce the running time of the algorithm of the present invention on very big instances. These heuristics now will be described.

When handling very large connected components (say, of size 100,000), computing a minimum weight cut is very costly. Moreover, large connected components often are rather sparse in the graphs that are encountered in practice and hence contain low weight vertices. Removing a minimum cut from such a component generally separates a low weight vertex, or a few such vertices, from the rest of the graph. This is computationally very expensive and not informative at an early stage of the clustering.

To overcome this problem, low weight vertices are screened from large components, prior to their clustering. The screening is done as follows: First the average vertex weight W in the component is computed, and is multiplied by a factor which is proportional to the natural logarithm of the size of the component. The resulting threshold is denoted by T*. Vertices whose weight is below T* then are removed, while updating the weight of the remaining vertices, until the updated weight of every (remaining) vertex is greater than T*. The removed vertices are marked as singletons and handled at a later stage.

Most preferably, the present invention uses the algorithm of J. Hao and J. Orlin, “A faster algorithm for finding the minimum cut in a directed graph”, Journal of Algorithms vol. 17 no. 3 pp. 424-446 (1994) for computing a minimum weight cut. This algorithm has been shown to outperform other minimum cut algorithms in practice. Its running time using highest label selection (C. Chekuri et al., “Experimental study of minimum cut algorithms”, Proceedings of the Eighth Annual ACM-SIAM Symposium on Discrete Algorithms, pp. 324-333 (1997)) is O(n²√m), where m is the number of edges. For large components this is computationally quite expensive. Thus, for components of size greater than 1,000 a different strategy is used. This strategy has been found to work in practice almost as well as computing a minimum cut.

The idea is to compute a minimum s-t cut in the underlying unweighted graph (where the weight of each edge is one), instead of a minimum weight cut. The complexity of this computation using Dinic's algorithm (S. Even, Graph Algorithms, Computer Science Press, Rockville Md. 1979, p. 119) is only O(nm^{2/3}) time. In order to use this approach, the vertices to be separated, s and t, are chosen so that their distance equals the diameter of the graph. More accurately, the diameter d of the graph is first computed, using breadth first search. If d≧4, two vertices s and t whose distance is d are chosen, and the graph is partitioned according to a minimum s-t cut.

Most preferably, the optimum thresholds for the edge weights, for the adoption step and for the merging step are determined heuristically. Different solutions, obtained using different thresholds, are compared using a likelihood score. If C is a suggested clustering solution, then the score of C is:

$$S ( C ) = {\sum }_{i , j mates in C}^{} ln \frac{f ( {S}_{ij} \vert i , j mates )}{f ( {S}_{ij} \vert i , j non - mates )} + {\sum }_{i , j non - mates in C}^{} ln \frac{f ( {S}_{ij} \vert i , j non - mates )}{f ( {S}_{ij} \vert i , j mates )}$$

According to the present invention, the quality of the solution is evaluated by computing two figures of merit to measure the homogeneity and separation of the produced clusters. For fingerprint data, homogeneity is evaluated by the average and minimum correlation coefficient between the fingerprint of an element and the fingerprint of its corresponding cluster. Precisely, if cl(u) is the cluster of u, F(X) and F(u) are the fingerprints of a cluster X and an element u, respectively, and S(x,y) is the correlation coefficient (or any other similarity measure) of fingerprints x and y, then

$$\begin{matrix}{H}_{Ave} = \frac{1}{N} {\sum }_{u ∈ N}^{} S ( F ( u ) , F ( cl ( u ) ) ) \\ {H}_{Min} = {min}_{u ∈ N} S ( F ( u ) , F ( cl ( u ) ) )\end{matrix}$$

Separation is evaluated by the weighted average and the maximum correlation coefficient between cluster fingerprints. That is, if the clusters are X₁, . . . X₁, then

$$\begin{matrix}{S}_{Ave} = \frac{1}{{\sum }_{i \neq j}^{} \vert {X}_{i} ‖ {X}_{j} \vert} {\sum }_{i \neq j}^{} \vert {X}_{i} ‖ {X}_{j} \vert S ( F ( {X}_{i} ) , F ( {X}_{j} ) ) \\ {S}_{Max} = {max}_{i \neq j} S ( F ( {X}_{i} ) , F ( {X}_{j} ) )\end{matrix}$$

Hence, a solution improves if H_{Ave} increases and H_{Min} increases, and if S_{Ave} decreases and S_{Max} decreases.

Applications

In the following, the results of applying the algorithm of the present invention to several data sets are described.

Gene Expression

The algorithm of the present invention first was tested on the yeast cell cycle dataset of R. Cho et al., “a genome-wide transcriptional analysis of the mitotic cell cycle, Mol. Cell vol. 2 pp. 65-73 (1998). That study monitored the expression levels of 6,218 S. cerevisiae putative gene transcripts (identified as ORFs) measured at ten minute intervals over two cell cycles (160 minutes). The results of the algorithm of the present invention were compared to those of the program GENECLUSTER (P. Tamao et al., “Interpreting patterns of gene expression with self-organizing maps: methods and application to hematopoietic differentiation”, PNAS vol. 96 pp. 2907-2912 (1999)) that uses Self-Organizing Maps. To this end, the same filtering and data normalization procedures of Tamao et al. were applied. The filtering removes genes which do not change significantly across samples, resulting in a set of 826 genes. The data preprocessing includes the removal of the 90-minutes time-point and normalizing the expression levels of each gene to have mean zero and variance one within each of the two cell-cycles.

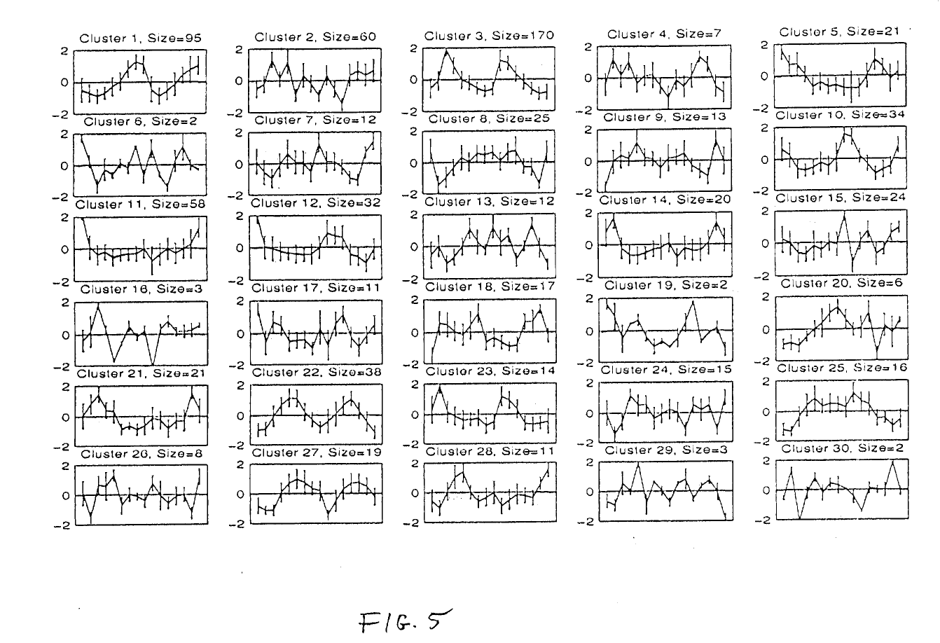

The algorithm of the present invention clustered the genes into 30 clusters. These clusters are shown in FIG. 5. In each plot, the x-axis is time points 0-80 and 100-160 at ten minute intervals and the y-axis is normalized expression levels. The solid lines are plots of the average patterns of the respective clusters. The error bars are the measured standard deviations. A summary of the homogeneity and separation parameters for the solutions produced by the algorithm of the present invention and by GENECLUSTER is shown in the following table.

| | | Homogeneity | | Separation | |
|---|---|---|---|---|---|
| Algorithm | No. clusters | H_{Ave} | H_{Min} | S_{Ave} | S_{Min} |
| present invention | 30 | 0.8 | −0.19 | −0.07 | 0.65 |
| GENECLUSTER | 30 | 0.74 | −0.88 | −0.02 | 0.97 |

The present invention obtained results superior in all the measured parameters. Two putative true clusters are the sets of late G1-peaking genes and M-peaking genes, reported by Cho et al. Out of the late G1-peaking genes that passed the filtering, the present invention placed 91% in a single cluster (see FIG. 5, cluster 3). In contrast, Tamayo et al. report that in their solution 87% of these genes were contained in three clusters. Out of the M-peaking genes that passed the filtering, the present invention placed 95% in a single cluster (see FIG. 5, cluster 1).

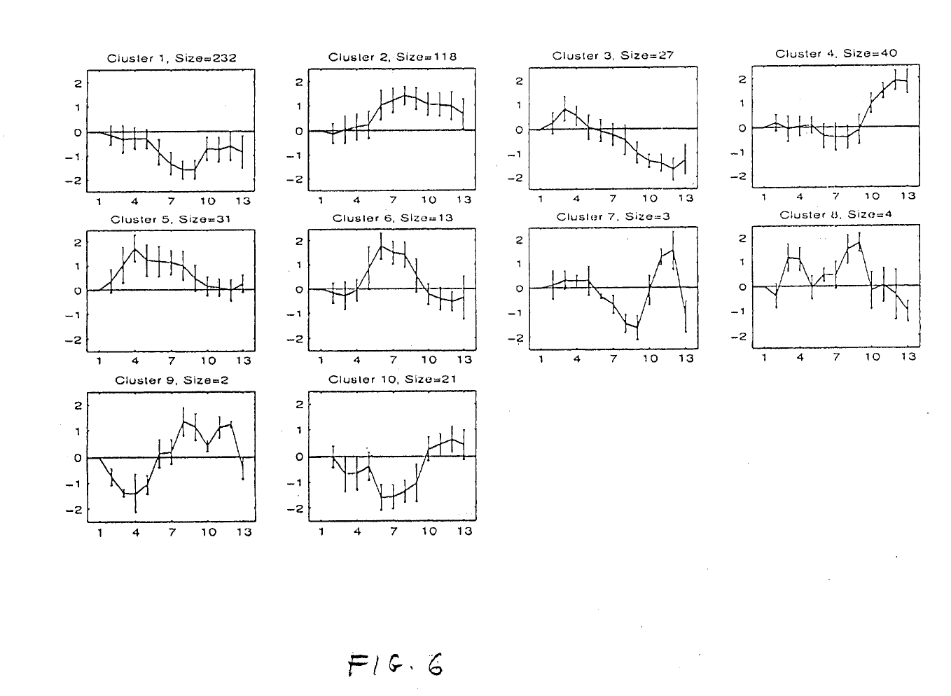

The second test of the algorithm of the present invention was an analysis of the dataset of V. lyer et al., “The transcriptional program in the response of human fibroblasts to serum”, Science vol. 283 no. 1 pp. 83-87 (1999), who studied the response of human fibroblasts to serum. This dataset contains expression levels of 8,613 human genes obtained as follows: Human fibroblasts were deprived of serum for 48 hours and then stimulated by addition of serum. Expression levels of genes were measured at 12 time-points after the stimulation. An additional data-point was obtained from a separate unsynchronized sample. A subset of 517 genes whose expression levels changed substantially across samples was analyzed by the hierarchical clustering method of Eisen et al. The data was normalized by dividing each entry by the expression level at time zero, and taking a natural logarithm of the result. For ease of manipulation, each fingerprint was transformed to have a fixed norm. The similarity function used was inner product, giving values identical to those used by Eisen et al. The present invention clustered the genes into 10 clusters. These clusters are shown in FIG. 6 In each plot, points 1-12 on the x-axis are synchronized time points, point 13 on the x-axis is an unsynchronized point, and the y-axis is normalized expression level. As in FIG. 5, the solid lines are plots of the average patterns of the respective clusters and the error bars are the measured standard deviations. The following table presents a comparison between the clustering quality of the present invention and the hierarchical clustering of Eisen et al. on this dataset.

| | | Homogeneity | | Separation | |
|---|---|---|---|---|---|
| Algorithm | No. clusters | H_{Ave} | H_{Min} | S_{Ave} | S_{Min} |
| present invention | 10 | 0.88 | 0.13 | −0.34 | 0.65 |
| hierarchical | 10 | 0.87 | −0.75 | −0.13 | 0.9 |

Again, the present invention performs better in all parameters.

The next two datasets studied were datasets of oligonucleotide fingerprints of cDNAs obtained from Max Planck Institute of Molecular Genetics in Berlin.

The first oligonucleotide fingerprint dataset contained 2,329 cDNAs fingerprinted using 139 oligonucleotides. This dataset was part of a library of some 100,000 cDNAs prepared from purified peripheral blood monocytes by the Novartis Forschungsinstitut in Vienna, Austria. The true clustering of these 2,329 CDNAs is known from back hybridization experiments performed with long, gene-specific oligonucleotides. This dataset contains 18 gene clusters varying in size from 709 to 1. The second oligonucleotide fingerprint dataset contains 20,275 cDNAs originating from sea urchin egg, fingerprinted using 217 oligonucleotides. For this dataset the true solution is known on a subset of 1,811 cDNAs. Fingerprint normalization was done as explained in S. Meier-Ewert et al., “Comparative gene expression profiling by oligonucleotide fingerprinting”, Genomics vol. 59 pp. 122-133 (1999).

Similarity values (inner products) between pairs of cDNA fingerprints from s5 the blood monocytes dataset are plotted in FIG. 7 To test the hypotheses that the distributions of the similarity values between mates and between non-mates are normal, a Kolmogorov-Smimov test was applied with a significance level of 0.05The hypotheses were accepted for both distributions, with the hypothesis regarding the non-mates distribution being accepted with higher confidence.

The following table shows a comparison of the results of the present invention on the blood monocytes dataset with those of the algorithm of Hartuv et al. In this table, “Minkowski” and “Jaccard” refer to two prior art figures of merit, the Minkowski measure (R. R. Sokal, “Clustering and classification: background and current directions”, in Classification and Clustering (J. Van Ryzin, ed., Academic Press, 1977) pp. 1-15) and the Jaccard coefficient (B. Everitt, Cluster Analysis (London: Edward Arnold, third edition, 1993)).

| | no. | no. | | | Time |
|---|---|---|---|---|---|
| algorithm | clusters | singletons | Minkowski | Jaccard | (min.) |
| present | 31 | 46 | 0.57 | 0.7 | 0.8 |
| invention | | | | | |
| Hartuv et al. | 16 | 206 | 0.71 | 0.55 | 43 |

The following table shows a comparison of the results of the present algorithm on the sea urchin dataset with those of the K-means algorithm Herwig et al.

| | no. | no. | | |
|---|---|---|---|---|
| algorithm | clusters | singletons | Minkowski | Jaccard |
| present | 2,952 | 1,295 | 0.59 | 0.69 |
| invention | | | | |
| Herwig et al. | 3,486 | 2,473 | 0.79 | 0.4 |

The present invention outperforms the other algorithms in all figures of merit, and also obtains solutions with fewer unclustered singletons.

The present invention was also applied to two protein similarity datasets. The first dataset contains 72,623 proteins from the ProtoMap project (G. Yona et al., “Protomap: automatic classification of protein sequences, a hierarchy of protein families, and local maps of the protein space”, Proteins: Structure, Function and Genetics vol. 37 pp. 360-378 (1999)). The second originated from the SYSTERS project (A. Krause et al., “The SYSTERS protein sequence cluster data set”, Nucleic Acid Research vol. 28 no. 1 (2000) pp. 270-272) and contains 117,835 proteins. Both datasets contain for each pair of proteins an E-value of their similarity as computed by BLAST.

Protein classification is inherently hierarchical, so the assumption of normal distribution of mate similarity values does not seem to hold. In order to apply the present invention to the data, the following modifications were made:

1. The weight of an edge (i,j) was set to be

$${w}_{ij} = ln \frac{{p}_{mates} ( 1 - {p}_{ij} )}{( 1 - {p}_{mates} ) {p}_{ij}} ,$$

where p_{ij is the E-value, and hence, also practically the p-value, of the similarity between i and j. A similarity threshold was used which corresponds to an E-value of} 10⁻²⁰.

2. For a minimum weight cut Γ which partitions G into H and H′,

$$W * ( Γ ) = ( r - \vert Γ \vert ) ln \frac{{p}_{mates}}{1 - {p}_{mates}} ,$$

was used, where r=|H∥H′|

3. For the adoption step, for each singleton r and each kernel K (considering the set of singletons R as an additional kernel), the ratio

$$\frac{{\sum }_{k ∈ K}^{} {w}_{rk}}{\vert K \vert}$$

was calculated. The pair r, K with the highest ratio was identified, and r was adopted to K if this ratio exceeded some predefined threshold w*.

4. For the merging step, for each pair of kernels K₁ and K₂ the ratio

$$\frac{{\sum }_{{k}_{1} ∈ {K}_{1} , {k}_{2} ∈ {K}_{2}}^{} {w}_{{k}_{1} {k}_{2}}}{\vert {K}_{1} ‖ {K}_{2} \vert}$$

was calculated. The pair K₁,K₂ with the highest ratio was identified. K₁ and K₂ were merged if this ratio exceeded w*.

For the evaluation of the ProtoMap dataset, a Pfam classification was used, for a subset of the data consisting of 17,244 single-domain proteins, which is assumed to be the true solution for this subset. The results of the present invention were compared to the results of ProtoMap with a confidence level of 10⁻²⁰ on this dataset The comparison is shown in the following table

| | no. | no. | | |
|---|---|---|---|---|
| algorithm | clusters | singletons | Minkowski | Jaccard |
| present | 7,747 | 16,612 | 0.88 | 0.39 |
| invention | | | | |
| ProtoMap | 7,445 | 16,408 | 0.89 | 0.39 |

The results are very similar, with a slight advantage to the present invention.

For the SYSTERS dataset, no “true solution” was assumed, so the solutions of CLICK and SYSTERS were evaluated using the figures of merit described in R. Sharan and R. Shamir, “CLICK: a clustering algorithm with applications to gene expression analysis”, Proceedings of the Eighth International Conference on Intelligent Systems for Molecular Biology (ISMB 2000), pp. 307-316. The following table presents the results of the comparison.

| | no. | no. | | |
|---|---|---|---|---|
| algorithm | clusters | singletons | Homogeneity | Separation |
| present | 9,429 | 17,119 | 0.24 | 0.03 |
| invention | | | | |
| SYSTERS | 10,891 | 28,300 | 0.14 | 0.03 |

The results show a significant advantage to the present invention.

For the above examples, the algorithm of the present invention was coded in C and executed on an SGI ORIGIN200 machine utilizing one IP27 processor. The implementation uses, in practice, linear space, and stores the similarity graph in a compact form by using linked adjacency lists. The following table summarizes the time performance of the algorithm of the present invention on the datasets described above.

| No. elements | No. edges | Time (min.) |
|---|---|---|
| 517 | 22,084 | 0.5 |
| 826 | 10,978 | 0.2 |
| 2,329 | 134,352 | 0.8 |
| 20,275 | 303,492 | 32.5 |
| 72,623 | 1,043,937 | 53 |
| 117,835 | 4,614,038 | 126.3 |

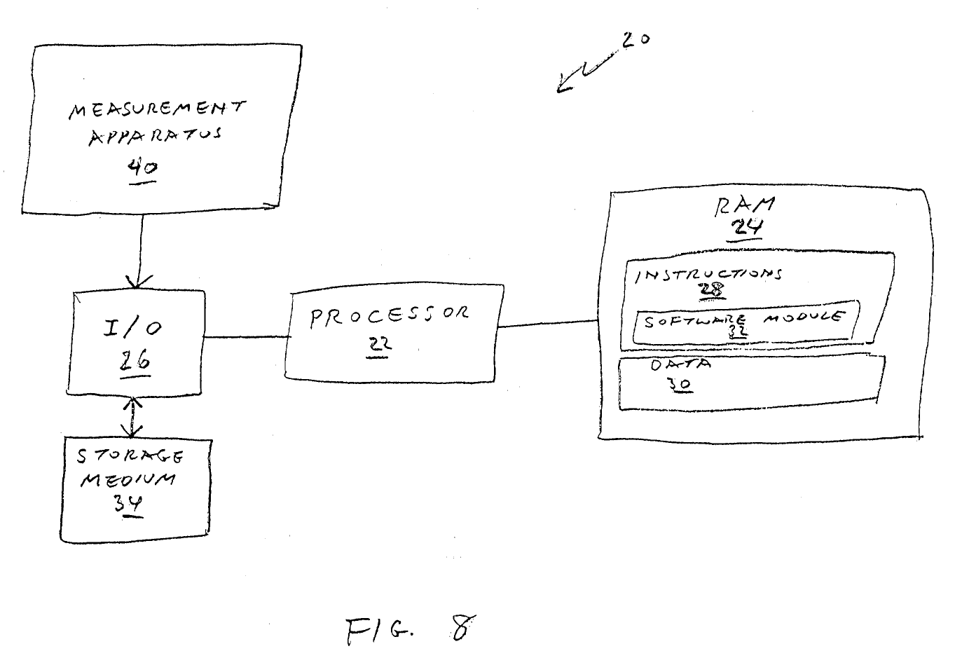

FIG. 8 is a high level block diagram of a system 20 for gathering and clustering gene expression data (or other data) according to the present invention. System 20 includes a processor 22, a random access memory 24 and a set of input/output devices, such as a keyboard, a floppy disk drive, a printer and a video monitor, represented by I/O block 26. Memory 24 includes an instruction storage area 28 and a data storage area 30. Within instruction storage area 28 is a software module 32 including a set of instructions which, when executed by processor 22, enable processor 22 to classify gene expression data by the method of the present invention.

The source code of software module 32 is provided on a suitable storage medium 34, such as a floppy disk or a compact disk. This source code is coded in a suitable high-level language. Selecting a suitable language for the instructions of software module 32 is easily done by one ordinarily skilled in the art. The language selected should be compatible with the hardware of system 20, including processor 22, and with the operating system of system 20. Examples of suitable languages include but are not limited to compiled languages such as FORTRAN, C and C++. Processor 22 reads the source code from storage medium 34, using a suitable input device 26, and stores the source code in software module 32.

If a compiled language is selected, a suitable compiler is loaded into instruction storage area 28. Following the instructions of the compiler, processor 22 turns the source code into machine-language instructions, which also are stored in instruction storage area 28 and which also constitute a portion of software module 32. The gene expression data to be clustered is entered via a suitable input device 26, either from a storage medium similar to storage medium 34, or directly from a gene expression measurement apparatus 40. Apparati for measuring gene expression are well known in the art and need not be detailed here. See, for example, U.S. Pat. No. 6,040,138, to Lockhart et al., and U.S. Pat. No. 6,156,502, to Beattie, both of which patents are incorporated by reference for all purposes as if fully set forth herein. Alternatively, if processor 22 is used to control apparatus 40, then the gene expression data to be clustered are provided directly to processor 22 by apparatus 40. In either case, processor 22 stores the gene expression data in data storage area 30.

Following the machine-language instructions in instruction storage area 28, processor 22 clusters the gene expression data according to the principles of the present invention. If the gene expression data are in the form of similarity values, processor 22 constructs a graph, each of whose edges is weighted according to the similarity value of the two genes that correspond to the two vertices connected by that edge. Processor 22 then partitions the graph into kernels and merges the kernels into clusters. If the gene expression data are in the form of fingerprints, processor 22 first computes similarity values from the fingerprints. The outcome of the clustering is displayed at video monitor 26 or printed on printer 26, preferably in graphical form as in FIGS. 5-7. In addition, the homogeneity and separation figures of merit are displayed.

While the invention has been described with respect to a limited number of embodiments, it will be appreciated that many variations, modifications and other applications of the invention may be made.

What is claimed is:

1. A method of classifying a plurality of elements, comprising the steps of:
(a) for each pair of elements, measuring a respective similarity value;
(b) partitioning a graph, each vertex whereof corresponds to a respective element, and each edge whereof is assigned a superbinary weight that is based on said similarity value of said pair of elements corresponding to said vertices that are connected by said each edge, into a plurality of kernels, according to said weights; and
(c) merging said kernels into clusters.

2. The method of claim 1, further comprising the step of:
(d) subsequent to said merging, for at least one of said vertices that is a singleton, adopting each said at least one singleton into a respective one of said clusters.

3. The method of claim 2, wherein said adopting is effected only if a similarity of said at least one singleton to said respective cluster exceeds a predefined threshold.

4. The method of claim 1, wherein said superbinary weights are based on said similarity values according to a probabilistic model.

5. The method of claim 4, wherein said probabilistic model assumes that said similarity values are distributed according to at least one probability distribution.

6. The method of claim 5, wherein said at least one probability distribution is a normal probability distribution.

7. The method of claim 5, wherein said probabilistic model assumes that said similarity values are distributed according to a first probability distribution for mates and according to a second probability distribution for non-mates.

8. The method of claim 7, wherein, for each cut of each said kernel, a probability that said each cut includes only mates exceeds a probability that said each cut includes only non-mates.

9. The method of claim 4, further comprising the step of:
(d) estimating at least one parameter of said probabilistic model, prior to said partitioning.

10. The method of claim 9, wherein said estimating is based on a previously classified subplurality of the elements.

11. The method of claim 9, wherein said estimating is effected using an EM algorithm.

12. The method of claim 1, wherein said graph includes at least one composite connected component, and wherein said partitioning includes the step of effecting a bipartition of each said at least one composite connected component of said graph.

13. The method of claim 12, wherein at least one of said at least one bipartition is effected as a minimum weight cut.

14. The method of claim 12, wherein at least one of said at least one bipartition is effected as a minimum s-t cut.

15. The method of claim 12, wherein said partitioning includes: subsequent to said at least one bipartition, for at least one of said vertices that is a singleton adopting each said at least one vertex into a respective one of said kernels.

16. The method of claim 5, wherein said adopting is effected only if a similarity of said at least one singleton to said respective kernel exceeds a predefined threshold.

17. The method of claim 11, wherein said partitioning includes: prior to said at least one bipartition, for at least one said composite connected component, optionally screening at least one vertex of said at least one composite connected component.

18. The method of claim 1, wherein said measuring of said similarity value is effected by steps including:
(i) for each element of said each pair of elements, measuring a fingerprint of said each element; and
(ii) computing said similarity value from said fingerprints.

19. The method of claim 18, wherein said computing is effected by steps including: for each said pair of elements: taking an inner product of said fingerprints of said each pair of elements.

20. A system for classifying a plurality of elements, comprising:
(a) an apparatus for measuring, for each pair of elements, a corresponding similarity value;
(b) a memory for storing said similarity values; and
(c) a processor for:
(i) partitioning a graph, each vertex whereof corresponds to a respective element, and each edge whereof is assigned a superbinary weight that is based on said similarity value of said pair of elements corresponding to said vertices that are connected by said each edge, into a plurality of kernels, according to said weights, and
(ii) merging said kernels into clusters.

21. A system for classifying a plurality of elements, comprising:
(a) an apparatus for measuring, for each element, a respective fingerprint;
(b) a memory for storing said fingerprints; and
(c) a processor for:
(i) computing, for each pair of elements, from said fingerprints thereof, a corresponding similarity value,
(ii) partitioning a graph, each vertex whereof corresponds to a respective element, and each edge whereof is assigned a superbinary weight that is based on said similarity value of said pair of elements corresponding to said vertices that are connected by said each edge, into a plurality of kernels, according to said weights, and
(iii) merging said kernels into clusters.

22. A method for analyzing signals containing a data set which is representative of a plurality of physical phenomena, to identify and distinguish among the physical phenomena by determining clusters of data points within the data set, the method comprising the steps of:
(a) associating a similarity value with each pair of data points;
(b) partitioning a graph, each vertex whereof corresponds to a respective data point, and each edge whereof is assigned a superbinary weight that is based on said similarity value of said pair of data points corresponding to said vertices that are connected by said each edge, into a plurality of kernels, according to said weights;
(c) merging said kernels to form the clusters; and
(d) identifying the physical phenomena based on the data clusters.

23. An apparatus for analyzing signals containing a data set which is representative of a plurality of physical phenomena, to identify and distinguish among the physical phenomena by determining clusters of data points within the data set, comprising:
(a) a mechanism for associating, with each pair of data points, a corresponding similarity value;
(b) a mechanism for partitioning a graph, each vertex whereof corresponds to a respective data point, and each edge whereof is assigned a superbinary weight that is based on said similarity value of said pair of data points corresponding to said vertices that are connected by said each edge, into a plurality of kernels according to said weights; and
(c) a mechanism for merging said kernels to form the clusters.

24. The apparatus of claim 23, further comprising:
(d) a memory for storing said data set and for providing the signals to the mechanisms.

25. A computer readable storage medium having computer readable code embodied on said computer readable storage medium, the computer readable code for clustering multi-dimensional related data in a computer database, the computer database including a set of data records, each data record storing information about a respective object of interest, the computer readable code comprising:
(a) program code for computing a similarity value for each pair of data records;
(b) program code for constructing a graph, each vertex whereof corresponds to a respective data record, and each edge whereof is assigned a superbinary weight that is based on said similarity value of said pair of data records corresponding to said vertices that are connected by said each edge;
(c) program code for partitioning said graph into a plurality of kernels according to said weights; and
(d) program code for merging said kernels to form the clusters.

26. A computer readable storage medium having computer readable code embodied on said computer readable storage medium, the computer readable code for clustering multi-dimensional related data in a computer database, the computer database including a set of data records, each data record storing information about a respective object of interest, the computer database also including, for at least one pair of data records, a corresponding similarity value, the computer readable code comprising:
(a) program code for constructing a graph, each vertex whereof corresponds to a respective data record, and each edge whereof is assigned a superbinary weight that is based on the similarity value of said pair of data records corresponding to said vertices that are connected by said each edge;
(b) program code for partitioning said graph into a plurality of kernels according to said weights; and
(c) program code for merging said kernels to form the clusters.

27. A method of classifying a plurality of elements, comprising the steps of:
(a) for each pair of elements, measuring a respective similarity value;
(b) partitioning the elements into clusters according to said similarity values;
(c) computing at least one figure of merit, for said partitioning, selected from the group consisting of:
(i) at least one measure of a homogeneity of said clusters, and
(ii) at least one measure of a separation of said clusters.

28. The method of claim 27, wherein said measuring of said similarity value is effected by steps including:
(i) for each element of said each pair of elements, measuring a fingerprint of said each element; and
(ii) computing said similarity value from said fingerprints;

29. The method of claim 28, wherein said at least one measure of said homogeneity includes an average of similarity measures of said fingerprints of the elements and respective fingerprints of said clusters thereof.

30. The method of claim 29, wherein said similarity measure is a correlation coefficient.

31. The method of claim 28, wherein said at least one measure of said homogeneity includes a minimum similarity measure of said fingerprints of the elements and respective fingerprints of said clusters thereof.

32. The method of claim 31, wherein said similarity measure is a correlation coefficient.

33. The method of claim 28, wherein said at least one measure of said separation includes a weighted average of similarity measures of fingerprints of said clusters.

34. The method of claim 33, wherein said similarity measure is a correlation coefficient.

35. The method of claim 28, wherein said at least one measure of said homogeneity includes a maximum similarity measure of fingerprints of said clusters.

36. The method of claim 35, wherein said similarity measure is a correlation coefficient.

37. A method for analyzing signals containing a data set which is representative of a plurality of physical phenomena, to identify and distinguish among the physical phenomena by determining clusters of data points within the data set, the method comprising the steps of:
(a) for each pair of elements, measuring a respective similarity value;
(b) partitioning the elements into clusters according to said similarity values;
(c) computing at least one figure of merit, for said partitioning, selected from the group consisting of:
(i) at least one measure of a homogeneity of said clusters; and
(ii) at least one measure of a separation of said clusters, and
(d) identifying the physical phenomena based on the data clusters.
